(19) United States
(12) Patent Application Publication
(10) Pub. No.: US 2026/0259399 A1
(43) Pub. Date: Sep. 3, 2026

(54) PORTABLE AND LOW-COST MICROSCOPIC IMAGING DEVICE

(71) Applicant: Vital Biosciences Inc., Mississauga (CA)

(72) Inventors: Iman Khodadadzadeh, San Carlos, CA (US); Eric James Mahoney, Hamilton (CA)

(21) Appl. No.: 19/162,262

(22) PCT Filed: Mar. 8, 2024
(86) PCT No.: PCT/IB2024/000160

§ 371 Date: Sep. 4, 2025

Related U.S. Application Data
(60) Provisional application No. 63/488,992, filed on Mar. 8, 2023.

(51) Int. Cl. G02B 21/04; G01N 33/49; G01N 33/493; G02B 5/02; G02B 21/02; G02B 21/06; G02B 21/18; G02B 21/24; G02B 21/26; G02B 27/10

(52) U.S. Cl. CPC G02B 21/04; G02B 5/0278; G02B 21/025; G02B 21/06; G02B 21/18; G02B 21/241; G02B 21/26; G02B 27/1013; G01N 33/49; G01N 33/493

(57) ABSTRACT

Microscopic imaging devices, systems and methods are provided. A microscopic imaging device includes a lens assembly, a first light source assembly, and one or more detection assemblies. The lens assembly has relatively short working distance, a relatively light weight, and/or multiple optical elements. The first light source assembly is configured to illuminate and/or excite a sample disposed at the working distance of the lens assembly. The one or more detection assemblies are configured to detect the light from the sample. The lens assembly, the first light source assembly, and at least one detection assembly are positioned to form an epiconfiguration.

### CROSS-REFERENCE TO RELATED APPLICATION(S)

The present application claims priority to U.S. Provisional Patent Application No. 63/488,992 filed Mar. 8, 2023. The disclosure of the application is incorporated herein for all purposes by reference in its entirety.

### TECHNICAL FIELD

The present disclosure relates to imaging devices, systems and methods. In particular, the present disclosure relates to small, lightweight and low-cost microscopic imaging devices, systems having microscopic imaging devices, and methods of using microscopic imaging devices and systems for analyzing biological samples.

### BACKGROUND

In microscopy, objective lenses are usually designed to provide long focus distances and large human views. A typical microscope lens costs at least a few hundred dollars. A typical microscope lens also weighs about 50 grams or more, and thus requires a certain amount of actuation force to move it (e.g., to tune the focus). Components that can provide the required force, such as voice coil motors, are often large and expensive. In some cases, a voice coil motor alone can cost thousands of dollars. As such, most existing microscopes are expensive and bulky, and are used in laboratories; they are generally not suitable or affordable for personal use at home, point-of-care applications, or the like.

### SUMMARY

Given the above background, there remains a need in the art for microscopic imaging devices, systems and methods that are suitable and affordable for use not only in hospitals/clinics but also at homes and in point-of-care applications. The present disclosure addresses these and other needs in the art by providing portable and affordable microscopic imaging devices, and by providing suitable systems and methods for analyzing biological samples such as body fluids or the like.

In one aspect, the present disclosure provides an imaging device that is small, light-weight and inexpensive. The imaging device includes a lens assembly, a first light source assembly, and one or more detection assemblies. The lens assembly includes a compound lens and is configured to be in optical communication with a sample chamber at a detection area. The lens assembly has (i) an optical path of less than about 15 mm, (ii) a working distance of no more than about 5 mm, (iii) a weight of no more than about 50 grams, (iv) more than three optical elements, or any combination thereof. The first light source assembly includes a first light source and is configured to emit light onto the sample chamber at the detection area. The first light source assembly is positioned on the same side of the sample chamber as the lens assembly. The one or more detection assemblies are in optical communication with the sample chamber at the detection area, and are configured to detect light traveling from the sample chamber at the detection area through the lens assembly. The imaging device is configured to define a first optical path in which light emitted from the first light source passes through the lens assembly and is directed onto the sample chamber at the detection area. The imaging device is also configured to define a second optical path in which light returning from the sample chamber at the detection area on the sample stage assembly passes through the lens assembly and at least a portion of the light returning from the sample chamber at the detection area is directed to the one or more detection assemblies.

In some embodiments, a first optical element in the more than three optical elements that is closest to an object side plane of the lens assembly has a first clear aperture diameter and is at a first distance relative to the object side plane of the lens assembly. A second optical element in the more than three optical elements that is farthest to the object side plane of the lens assembly has a second clear aperture diameter and is at a second distance relative to the object side plane of the lens assembly. The first clear aperture diameter is equal to or greater than the second clear aperture diameter, and the first distance is equal to or greater than 0.4 times of the second distance. In some embodiments, the first distance defines the working distance of the lens assembly.

In some embodiments, the working distance of the lens assembly is less than about 4 mm, less than 3 mm, less than 2 mm, or less than 1.5 mm. In some embodiments, the working distance of the lens assembly is about 1.0 mm, about 1.2 mm, about 1.4 mm or about 1.6 mm. In some embodiments, the imaging device has dimensions of less than 25 centimeters (cm) in length, less than 25 cm in width, and 25 cm in height.

In some embodiments, the compound lens of the lens assembly has an optical axis and an image surface through which the optical axis passes. The compound lens includes a plastic lens barrel surrounding the optical axis. The plastic lens barrel includes an image-side portion and an object-side aperture through which the optical axis passes. The imaging device is used such that the sample is located at the finite focal plane of the compound lens at a distance designated as its back focal length in designs. In some embodiments, the compound lens includes two imaging lenses with two different back focal lengths. The object-side uses the imaging lens with a shorter back focal length and the image-side uses the imaging lens with a longer back focal length. The effective magnification of the whole imaging device would be determined by the ratio of the back focal lengths of the two imaging lenses. This configuration would allow the use of a variety of imaging lens designs that could result in 1×, 2×, 5×, 10×, 15×, 20× or larger magnification factors. In some embodiments, the compound lens includes between 2 and 10 lenses. In some embodiments, the compound lens is a commodity lens reversely positioned such that the sample chamber is located at a back focal plane of the commodity lens.

In some embodiments, the first light source assembly is configured to emit light at a single narrow wavelength band. In some embodiments, the single narrow wavelength bands has a full width at half max (FWHM) of no more than 50 nm, no more than 25 nm, no more than 10 nm, or no more than 5 nm. In some embodiments, the first light source assembly includes one or more light-emitting diodes (LEDs). In some embodiments, one or more LEDs includes a first LED configured to emit light at a first narrow wavelength band and a second LED configured to emit light at a second narrow wavelength band. In an embodiment, the first light source is a single LED.

In some embodiments, the imaging device also includes a first dichroic mirror positioned in the first optical path between the lens assembly and the first light source assembly and positioned in the second optical path between the lens assembly and the one or more detection assemblies. The first dichroic mirror has a passband including either (a) an excitation wavelength band emitted by the first light source, or (b) one or more emission wavelength bands. The first dichroic mirror also has a stopband including the other of (a) the excitation wavelength band emitted by the first light source, or (b) the one or more emission wavelength bands. In an embodiment, the first dichroic mirror transmits the excitation light emitted by the first light source assembly to the lens assembly. The lens assembly directs the excitation light onto the area of the sample. The lens assembly collects the emission light emitted by the one or more components of the sample and passes the collected emission light to the first dichroic mirror. The first dichroic mirror reflects the emission light to a second optical path that differs from the first optical path. At least one of the one or more detection assemblies is in optical communication with the first dichroic mirror to detect the emission light reflected by the first dichroic mirror. In an alternative embodiment, the first dichroic mirror reflects the excitation light emitted by the first light source assembly to the lens assembly. The lens assembly directs the excitation light onto the area of the sample. The lens assembly collects the emission light emitted by the one or more components of the sample and passes the collected emission light to the first dichroic mirror. The first dichroic mirror transmits the emission light to a second optical path that differs from the first optical path. At least one of the one or more detection assemblies is in optical communication with the first dichroic mirror to detect the emission light transmitted by the first dichroic mirror.

In some embodiments, the imaging device includes a second dichroic mirror positioned in the second optical path between the first dichroic mirror and the one or more detection assemblies. The second dichroic mirror has a passband including a first portion of the one or more emission wavelength bands, and a stopband including a second portion of the one or more emission wavelength bands. The one or more detection assemblies include a first detection assembly and a second detection assembly. The first detection assembly includes a first two-dimensional optical detector configured to acquire an image at a waveband in the first portion of the one or more emission wavelength bands. The second detection assembly includes a second two-dimensional optical detector configured to acquire an image at a waveband in the second portion of the one or more emission wavelength bands. In some embodiments, the first detection assembly includes a first tube lens positioned between the second dichroic mirror and the first two-dimensional optical detector, and the second detection assembly includes a second tube lens positioned between the second dichroic mirror and the second two-dimensional optical detector.

In some embodiments, the second dichroic mirror is configured to transmit the emission light at a first wavelength range and reflect the emission light at a second wavelength range that differs from the first wavelength range. The one or more detection assemblies include a first detection assembly to detect the emission light at the first wavelength range and a second detection assembly to detect the emission light at the second wavelength range.

Alternatively, in some embodiments, the imaging device includes at least one splitter, and a plurality of filters. The one or more detection assemblies includes a plurality of detection assemblies. The at least one splitter is positioned in the second optical path between the first dichroic mirror and the plurality of detection assemblies and configured to split the one or more emission wavelength bands into a plurality of portions. Each respective filter in the plurality of filters is positioned between the at least one splitter and a corresponding detection assembly in the plurality of detection assemblies and has a respective passband in a plurality of passbands to filter a corresponding portion in the plurality of portions of the one or more emission wavelength bands. The corresponding detection assembly in the plurality of detection assemblies includes a two-dimensional optical detector configured to acquire an image of the corresponding portion in the plurality of portions of the one or more emission wavelength bands after it passes the respective filter in the plurality of filters.

In some embodiments, the imaging device includes a voice coil actuator coupled or integrated with the lens assembly to adjust a focus of the compound lens. In some embodiments, the voice coil actuator is smaller than 20 mm in width, 20 mm in length, and 20 mm in height. In some embodiments, the focus of the compound lens of the lens assembly is adjustable in a range of no more than about ±1.2 mm, about ±1.1 mm, about ±1.0 mm, about ±0.9 mm, or about ±0.8 mm.

In some embodiments, the imaging device includes a second light source assembly positioned on the opposite side of the sample stage assembly as the lens assembly. The second light source assembly is configured to emit light onto the sample chamber at the detection area. The imaging device is configured to define a third optical path in which light emitted from the second light source assembly passes through the sample chamber at the detection area and the lens assembly, and at least a portion of the light that passed through the sample chamber at the detection area is directed to the one or more detection assemblies. In some embodiments, the second light source assembly includes a brightfield light source, a darkfield light source, and/or a side scatter light source. The lens assembly is positioned to collect at least a portion of light scattered or transmitted by the sample and pass the collected light to the one or more detection assemblies. The one or more detection assemblies is configured to detect the light passed by the lens assembly.

In some embodiments, a control unit is configured to selectively activate the first light source assembly, the second light source assembly, any individual light source in the first light source assembly, any individual light source in the second light source assembly, or any combination thereof.

In some embodiments, the imaging device includes a sample stage assembly configured for positioning the sample chamber at the detection area. For instance, in some embodiments, the sample stage assembly is configured for moving the sample chamber with respect to the lens assembly. In some embodiments, the sample stage assembly includes a rotating spindle configured for rotating the sample chamber with respect to an optical axis of the lens assembly to enable a rotational field of view selection. In some embodiments, the sample stage assembly allows tilting and small variation of the sample in a focal plane of the lens assembly, and/or allows both sample preparation and processing. In some embodiments, the sample stage assembly is configured to move the sample chamber out of the detection area and position a different sample chamber at the detection area.

In some embodiments, the sample chamber is a portion of a centrifugal micro-fluidic biodisk, a portion of a capillary tube device, or a portion of a flow cell device. In some embodiments, the centrifugal micro-fluidic biodisk includes an elongated reservoir positioned radially about a rotational axis of the centrifugal micro-fluidic biodisk at a first distance from the rotational axis.

In another aspect, the present disclosure provides a system for analyzing biological samples. The system includes an imaging device, such as any imaging device disclosed herein or the like, that is configured to capture one or more images of the sample. The system also includes a control unit and a computing device. The control unit is in wire or wireless communication with the imaging device and configured to control the imaging device, e.g., to control the one or more light source assemblies, the one or more detection assemblies and/or other parts of the imaging device to perform a data or image capture procedure in a defined manner. The computing device is in wire or wireless communication with the control unit to process the one or more captured images of the sample.

In an embodiment, the control unit is a standalone unit. In another embodiment, the control unit is embedded or integrated with the imaging device. In still another embodiment, the control unit is embedded or integrated with the computing device.

In a further aspect, the present disclosure provides a method for analyzing biological samples. The method includes placing a sample at the working distance from a lens assembly of an imaging device, such as any imaging device disclosed herein or the like. The method also includes activating a first light source assembly of the imaging device to provide an excitation light to the sample, and detecting, by the one or more detection assemblies of the imaging device, the emission light emitted by the sample. In some embodiments, the method also includes processing the detected emission light to generate one or more sets of spectral and/or spatial responses, each set representing a spatial distribution of a corresponding component in the one or more components over the area of the sample. The processing is conducted at a computing device, such as the computing device disclosed herein or the like.

The devices, systems and methods of the present disclosure have other features and advantages that will be apparent from, or are set forth in more detail in, the accompanying drawings, which are incorporated herein, and the following Detailed Description, which together serve to explain certain principles of exemplary embodiments of the present disclosure.

### BRIEF DESCRIPTION OF THE DRAWINGS

The accompanying drawings, which are incorporated into and constitute a part of this specification, illustrate one or more exemplary embodiments of the present disclosure and, together with the Detailed Description, serve to explain the principles and implementations of exemplary embodiments of the invention. The accompanying drawings are not necessarily to scale. The specific design features of the present invention as disclosed herein, including, for example, specific dimensions, orientations, locations, and shapes will be determined in part by the particular intended application and use environment. In addition, the components illustrated in the figures are combinable in any useful number and combination.

In the drawings:

FIG. 1A is a schematic diagram illustrating a microscopic imaging device in accordance with some exemplary embodiments of the present disclosure;

FIG. 1B is a schematic diagram illustrating the microscopic imaging device of FIG. 1A including an optic assembly in accordance with an exemplary embodiment of the present disclosure;

FIG. 1C is a schematic diagram illustrating the microscopic imaging device of FIG. 1A including an optic assembly in accordance with an alternative exemplary embodiment of the present disclosure;

FIG. 2 is a schematic diagram illustrating a microscopic imaging device in accordance with some exemplary embodiments of the present disclosure;

FIG. 3 is a schematic diagram illustrating a microscopic imaging device in accordance with some exemplary embodiments of the present disclosure;

FIG. 4 is a schematic diagram illustrating a microscopic imaging device in accordance with some exemplary embodiments of the present disclosure;

FIG. 5 is a schematic diagram illustrating a microscopic system including a microscopic imaging device in accordance with some exemplary embodiments of the present disclosure;

FIG. 6 is a flow chart illustrating a method for analyzing a biological sample in accordance with some exemplary embodiments of the present disclosure;

FIG. 7 is a schematic diagram illustrating a lens assembly applicable to a microscopic imaging device in accordance with some exemplary embodiments of the present disclosure;

FIG. 8 is a schematic diagram illustrating a microscopic imaging device in accordance with some exemplary embodiments of the present disclosure; and

FIG. 9 is a schematic diagram illustrating a centrifugal micro-fluidic biodisk in accordance with some exemplary embodiments of the present disclosure.

### DETAILED DESCRIPTION

The present disclosure provides portable microscopic imaging devices, systems and methods for analyzing biological samples, such as body fluids (e.g., blood, plasma, urine) or the like. A microscopic imaging device of the present disclosure is generally small, light-weight, and less expensive. In certain embodiments, a microscopic imaging device of the present disclosure is ten to twenty folds smaller than existing microscopic imaging devices. A system of the present disclosure generally includes a microscopic imaging device in communication with a computing device. Accordingly, the devices, systems and methods provide a possibility for rapid screening, testing and medical diagnosis not only in hospitals/clinics but also at homes or other physical settings.

The reduction in size, weight and cost of the microscopic imaging devices is achieved in part by the configuration of the microscopic imaging devices. For instance, in certain embodiments, a microscopic imaging device of the present disclosure includes a lens assembly having a short working distance, one or more light source assemblies and one or more detection assemblies. The one or more light source assemblies are configured to illuminate and/or excite a sample disposed at the working distance of the lens assembly. The one or more detection assemblies are configured to detect the light from the sample, e.g., the light emitted by, reflected by, scattered by or transmitted through the sample. The lens assembly, at least one light source assembly, and at least one detection assembly are positioned to form an epi-configuration, i.e., both the light from the at least one light source assembly and the light from the sample to be detected by the at least one detection assembly travel through the same objective lens of the lens assembly.

The reduction in size, weight and cost of the microscopic imaging devices is also achieved in part by the use of commodity (e.g., cell-phone or consumer electronics) components. For instance, in certain embodiments, a microscopic imaging device of the present disclosure takes a commodity lens, reverses it and uses the reversed commodity lens as the objective lens of the microscopic imaging device. As a result, the objective lens of the microscopic imaging device of the present disclosure has a short working distance, usually less than 5 millimeters (mm), less than 4 mm, less than 3 mm, less than 2 mm, less than 1.5 mm, less than 1.4 mm, less than 1.3 mm, less than 1.2 mm, less than 1.1 mm, or less than 1.0 mm. Using a reversed commodity lens as the objective lens also allows for the use of other commodity components, resulting in further reduction in the overall size, weight and cost of the microscopic imaging device.

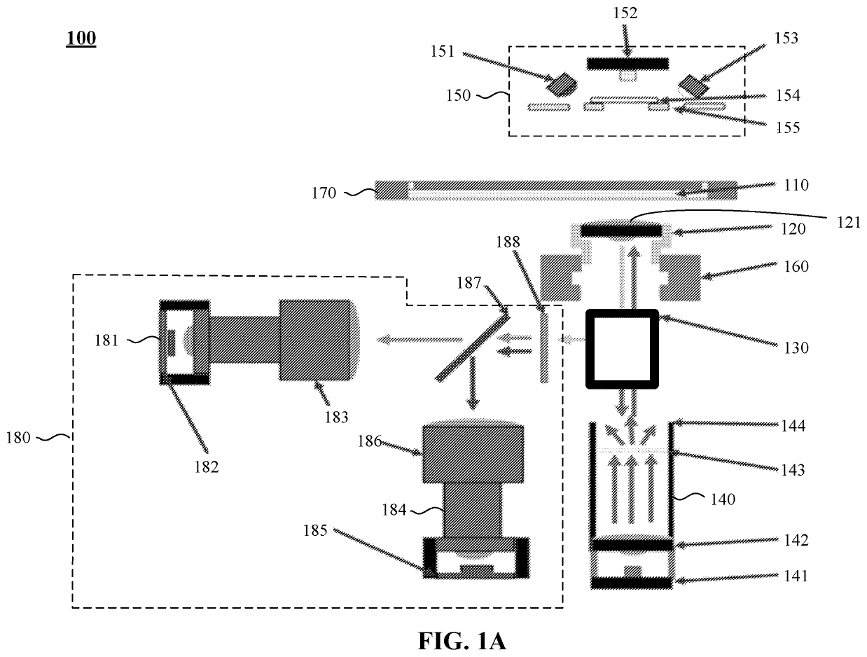

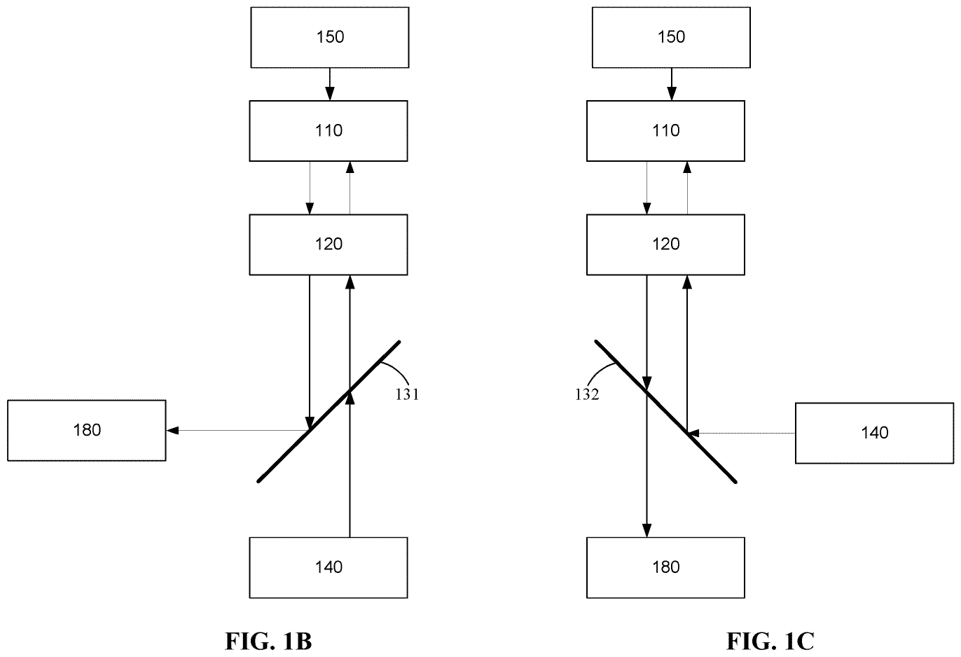

Referring now to the drawings, where like reference numerals indicate like elements throughout, there is shown in FIGS. 1A-1C an exemplary portable microscopic imaging device 100 in accordance with some embodiments of the present disclosure. The imaging device 100 includes a lens assembly 120, a light source assembly 140, and a detection unit 180. The light source assembly 140 is configured to emit a light when activated. The lens assembly 120 includes a compound lens configured to be in optical communication with a sample chamber 110 at a detection area (e.g., at a working distance of the lens assembly or a working distance of an objective lens 121 of the lens assembly). For instance, the lens assembly 120 is configured to receive the light emitted by the light source assembly 140 and direct the received light to the sample chamber 110 at the detection area. The lens assembly 120 is also configured to collect a light from a sample disposed in the sample chamber 110. The light collected from the sample includes light emitted by the sample, scattered by the sample, reflected by the sample, transmitted through the sample or any combination thereof. The lens assembly 120 is further configured to pass the collected light for detection by the detection unit 180.

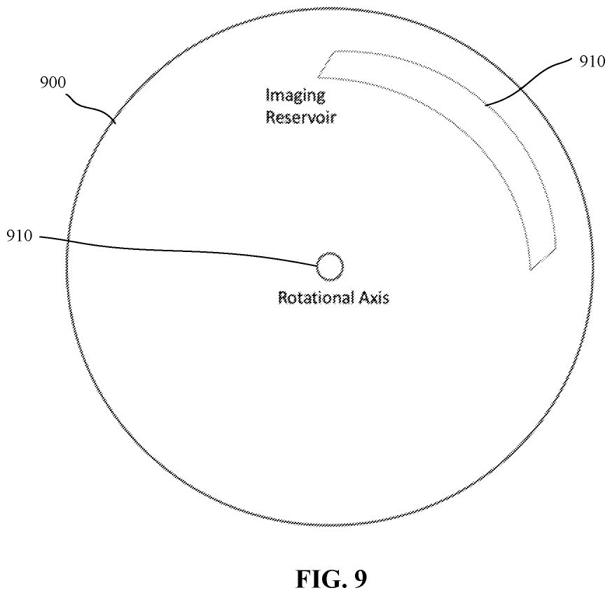

In some embodiments, the sample chamber is a portion of a centrifugal micro-fluidic biodisk, a portion of a capillary tube device, or a portion of a flow cell device. In some embodiments, the centrifugal micro-fluidic biodisk includes an elongated reservoir positioned radially about a rotational axis of the centrifugal micro-fluidic biodisk at a first distance from the rotational axis. As a non-limiting example, FIG. 9 illustrates an exemplary centrifugal micro-fluidic biodisk 900. In some embodiments, the centrifugal micro-fluidic biodisk 900 includes an elongated reservoir 910 positioned radially about a rotational axis of the centrifugal micro-fluidic biodisk at a first distance from the rotational axis 920.

In some embodiments, the imaging device 100 includes an optic assembly 130 in optical communication with the lens assembly 120, light source assembly 140 and detection unit 180. The optic assembly 130 is configured to relay the light emitted by the light source assembly 140 to the lens assembly 120 and relay the light from the sample collected and passed by the lens assembly 120 to the detection unit 180. For instance, in some embodiments, the optic assembly 130 includes a dichroic mirror that reflects a light at some wavelength(s) and transmits a light at some other wavelength(s). For instance, the dichroic mirror has a passband including either (a) an excitation wavelength band emitted by the first light source, or (b) one or more emission wavelength bands. The dichroic mirror also has a stopband including the other of (a) the excitation wavelength band emitted by the first light source, or (b) the one or more emission wavelength bands.

As a non-limiting example, FIG. 1B illustrates the optic assembly 130 including a dichroic mirror 131. The dichroic mirror 131 is disposed in an optical path between the lens assembly 120 and the light source assembly 140 and in an optical path between the lens assembly 120 and the detection assembly 180. In other words, the dichroic mirror 131 is in optical communication with the lens assembly, light source assembly and detection assembly. The optical path between the lens assembly and light source assembly is different than the optical path between the lens assembly and detection assembly. The dichroic mirror 131 transmits the light emitted by the first light source assembly (e.g., illumination and/or excitation light) to the lens assembly. The lens assembly directs the illumination and/or excitation light onto an area of the sample, which is placed at the working distance or focal plane of the lens assembly. The lens assembly collects the light from the sample (e.g., emitted by, scattered by, reflected by and/or transmitted through the sample), and passes the collected light to the dichroic mirror 131. The dichroic mirror 131 reflects the light collected and passed by the lens assembly to the detection assembly.

As another non-limiting example, FIG. 1C illustrates the optic assembly 130 including a dichroic mirror 132. Similar to the dichroic mirror 131, the dichroic mirror 132 is disposed in an optical path between the lens assembly and light source assembly and in an optical path between the lens assembly and detection assembly that is different than the optical path between the lens assembly and light source assembly. Unlike the dichroic mirror 131, the dichroic mirror 132 reflects the light emitted by the first light source assembly to the lens assembly and transmits the light collected and passed by the lens assembly to the detection assembly.

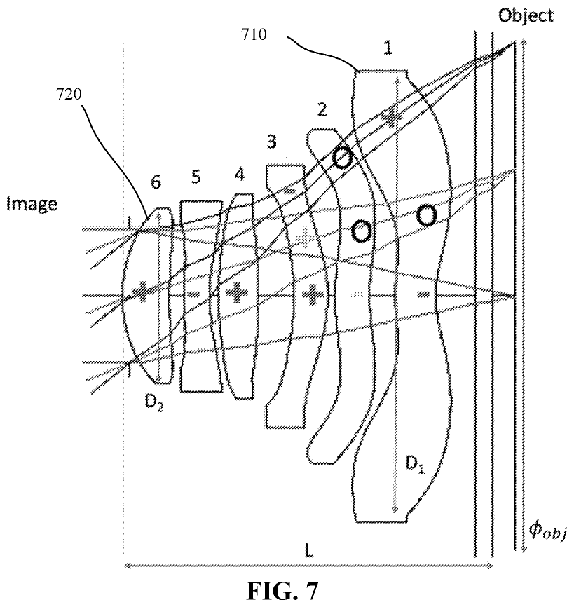

In various embodiments, the lens assembly 120 has (i) an optical path of less than about 15 mm, (ii) a working distance of no more than about 5 mm, (iii) a weight of no more than about 50 grams, (iv) more than three optical elements, or any combination thereof. For instance, as a non-limiting example, FIG. 7 illustrates the lens assembly 120 including more than three optical elements. Among the more than three optical elements, a first optical element 710 is closest to an object side plane of the lens assembly and a second optical element 720 is farthest to the object side plane of the lens assembly. The first optical element 710 has a first clear aperture diameter (generally designated as D1). The second optical element 720 has a second clear aperture diameter (generally designated as D2). The first clear aperture diameter D1 is equal to or greater than the second clear aperture diameter D2. For instance, in some embodiments, the ratio of D1/D2 is about 1, about 1.1, about 1.2, about 1.3, about 1.5, about 1.6, about 1.7, about 1.8, about 1.9, about 2, about 2.25, about 2.5, about 2.75, or about 3. In some embodiments, the ratio of D1/D2 is at least 1, at least 1.1, at least 1.2, at least 1.3, at least 1.4, at least 1.5, at least 2, at least 2.5, at least 3, at least 3.5, at least 4, at least 4.5, or at least 5.

In some embodiments, the ratio of D1/D2 is from 1 to 10, from 1 to 5, from 1 to 4, from 1 to 3, from 1 to 2.5, from 1 to 2.25, from 1 to 2, from 1 to 1.75, from 1 to 1.5, from 1 to 1.25, from 1 to 1.1, from 1.1 to 10, from 1.1 to 5, from 1.1 to 4, from 1.1 to 3, from 1.1 to 2.5, from 1.1 to 2.25, from 1.1 to 2, from 1.1 to 1.75, from 1.1 to 1.5, from 1.1 to 1.25, from 1.25 to 10, from 1.25 to 5, from 1.25 to 4, from 1.25 to 3, from 1.25 to 2.5, from 1.25 to 2.25, from 1.25 to 2, from 1.25 to 1.75, from 1.25 to 1.5, from 1.5 to 10, from 1.5 to 5, from 1.5 to 4, from 1.5 to 3, from 1.5 to 2.5, from 1.5 to 2.25, from 1.5 to 2, from 1.5 to 1.75, from 1.75 to 10, from 1.75 to 5, from 1.75 to 4, from 1.75 to 3, from 1.75 to 2.5, from 1.75 to 2.25, from 1.75 to 2, from 2 to 10, from 2 to 5, from 2 to 4, from 2 to 3, from 2 to 2.5, from 2 to 2.25, from 2.5 to 10, from 2.5 to 7.5 from 2.5 to 5, from 2.5 to 5, from 2.5 to 3, from 3 to 10, from 3 to 7.5, from 3 to 5, from 3 to 4, from 4 to 10, from 4 to 7.5, from 4 to 5, from 5 to 10, from 5 to 7.5, from 7.5 to 5, or some other range starting no lower than 1 and ending no higher than 10.

In some embodiments, the lens elements in front of (e.g., adjacent to) the object plane deviate significantly from the spherical shape. The refractive power of a lens in the center of the lens, whether positive or negative, is also shown. However, the first two lens elements have a strong field-dependent effect with (locally) very different refractive powers and deflections on the beam. The local curvature variation causes a change in the refractive power along each field-dependent light path. Positive (+), neutral (o), and negative (−) power is indicated in the figure for the field near lens elements 1, 2 and 3. This allows for better aberration correction as well as wider field of view for the objective lens in a more compact form factor. For instance, in some embodiments, the lens assembly 120 has an overall length (generally designated as L), and a diagonal dimension (Field number) at the object side plane of the lens assembly (generally designated as φ_{obj}). In some embodiments, the overall length L is about 5 mm to about 20 mm, and the diagonal dimension is about 3 mm to 20 mm.

The ratio of L/φ_{obj} defines a value (generally designated as r) as follows:

$$r = \frac{L}{{ϕ}_{obj}}$$

A compactness factor of a lens assembly may be calculated by multiplying the r value with the magnification of the lens assembly. As a non-limiting example, Table I below lists compactness factors for some exemplary lens assemblies with the overall length L of about 10 mm, where the diagonal dimension φ_{obj} is about 5.0 mm, about 5.8 mm, about 7.0 mm, about 8.0 mm, about 9.0 mm, or about 12.0 mm, and the magnification is 4, 8, 10, 20 or 40. As another non-limiting example, Table II below lists compactness factors for some exemplary lens assemblies with the overall length L of about 7 mm, where the diagonal dimension φ_{obj} is about 5.0 mm, about 5.8 mm, about 7.0 mm, about 8.0 mm, about 9.0 mm, or about 12.0 mm, and the magnification is 4, 8, 10, 20 or 40. In general, a lens assembly of the present invention has a compactness factor that is at least 20% smaller than that of a regular objective lens assembly. A regular objective lens assembly (e.g., a regular microscope objective) typically includes at least five lens components, which are divided into three groups: a front group, a middle group and a rear group in order from an object side. The front group has a positive refracting power as a whole and has a meniscus lens component with an object-side surface thereof being concave toward the object side. The middle group has a positive refracting power as a whole and has a plurality of cemented lens components. The rear group has a pair of concave air-contact surfaces arranged adjacent and opposite to one another. An example of a regular objective lens assembly is disclosed in U.S. Patent Application Publication No. US 2010/0165474 A1, the content of which is incorporated herein by reference in its entirety for all purposes. Different from the regular objective lens assemblies, the lens assembly of the present disclosure has a first group of one or more lenses in front of the object with variable refractive power across the radial direction from the optical axis (positive, negative or neutral). This allows for a wider field of view as well as smaller aberrations further away from the optical axis. Moreover, the overall length of the lens assembly of the present disclosure is relatively smaller, which directly leads to the smaller “r” value.

TABLE I
Compactness Factors (L = 10 mm)

Magnification

| φ_obj (mm diagonal) | 4 | 8 | 10 | 20 | 40 |
|---|---|---|---|---|---|
| 5.0 | 8.0 | 16.0 | 20.0 | 40.0 | 80.0 |
| 5.8 | 6.9 | 13.8 | 17.2 | 34.5 | 69.0 |
| 7.0 | 5.7 | 11.4 | 14.3 | 28.6 | 57.1 |
| 8.0 | 5.0 | 10.0 | 12.5 | 25.0 | 50.0 |
| 9.0 | 4.4 | 8.9 | 11.1 | 22.2 | 44.4 |
| 12.0 | 3.3 | 6.7 | 8.3 | 16.7 | 33.3 |

TABLE II
Compactness Factors (L = 7 mm)

Magnification

| φ_obj (mm diagonal) | 4 | 8 | 10 | 20 | 40 |
|---|---|---|---|---|---|
| 5.0 | 5.6 | 11.2 | 14.0 | 28.0 | 56.0 |
| 5.8 | 4.8 | 9.7 | 12.1 | 24.1 | 48.3 |
| 7.0 | 4.0 | 8.0 | 10.0 | 20.0 | 40.0 |
| 8.0 | 3.5 | 7.0 | 8.8 | 17.5 | 35.0 |
| 9.0 | 3.1 | 6.2 | 7.8 | 15.6 | 31.1 |
| 12.0 | 2.3 | 4.7 | 5.8 | 11.7 | 23.3 |

Accordingly, in some embodiments, a lens assembly used in an imaging device described herein has (i) an overall length of from 8 mm to 12 mm, a magnification of at least 4×, and a compactness factor of no more than 10. In some embodiments, a lens assembly used in an imaging device described herein has (i) an overall length of from 8 mm to 12 mm, a magnification of at least 4×, and a compactness factor of no more than 8. In some embodiments, a lens assembly used in an imaging device described herein has (i) an overall length of from 8 mm to 12 mm, a magnification of at least 4×, and a compactness factor of no more than 6. In some embodiments, a lens assembly used in an imaging device described herein has (i) an overall length of from 8 mm to 12 mm, a magnification of at least 4×, and a compactness factor of no more than 5. In some embodiments, a lens assembly used in an imaging device described herein has (i) an overall length of from 8 mm to 12 mm, a magnification of at least 4×, and a compactness factor of no more than 4. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Similarly, in some embodiments, a lens assembly used in an imaging device described herein has (i) an overall length of from 8 mm to 12 mm, a magnification of at least 8×, and a compactness factor of no more than 17.5. In some embodiments, a lens assembly used in an imaging device described herein has (i) an overall length of from 8 mm to 12 mm, a magnification of at least 8×, and a compactness factor of no more than 15. In some embodiments, a lens assembly used in an imaging device described herein has (i) an overall length of from 8 mm to 12 mm, a magnification of at least 8×, and a compactness factor of no more than 12.5. In some embodiments, a lens assembly used in an imaging device described herein has (i) an overall length of from 8 mm to 12 mm, a magnification of at least 8×, and a compactness factor of no more than 10. In some embodiments, a lens assembly used in an imaging device described herein has (i) an overall length of from 8 mm to 12 mm, a magnification of at least 8×, and a compactness factor of no more than 8. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Similarly, in some embodiments, a lens assembly used in an imaging device described herein has (i) an overall length of from 8 mm to 12 mm, a magnification of at least 10×, and a compactness factor of no more than 25. In some embodiments, a lens assembly used in an imaging device described herein has (i) an overall length of from 8 mm to 12 mm, a magnification of at least 10×, and a compactness factor of no more than 20. In some embodiments, a lens assembly used in an imaging device described herein has (i) an overall length of from 8 mm to 12 mm, a magnification of at least 10×, and a compactness factor of no more than 15. In some embodiments, a lens assembly used in an imaging device described herein has (i) an overall length of from 8 mm to 12 mm, a magnification of at least 10×, and a compactness factor of no more than 12.5. In some embodiments, a lens assembly used in an imaging device described herein has (i) an overall length of from 8 mm to 12 mm, a magnification of at least 10×, and a compactness factor of no more than 10. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Accordingly, in some embodiments, a lens assembly used in an imaging device described herein has (i) an overall length of from 8 mm to 12 mm, a magnification of at least 20×, and a compactness factor of no more than 40. In some embodiments, a lens assembly used in an imaging device described herein has (i) an overall length of from 8 mm to 12 mm, a magnification of at least 20×, and a compactness factor of no more than 30. In some embodiments, a lens assembly used in an imaging device described herein has (i) an overall length of from 8 mm to 12 mm, a magnification of at least 20×, and a compactness factor of no more than 25. In some embodiments, a lens assembly used in an imaging device described herein has (i) an overall length of from 8 mm to 12 mm, a magnification of at least 20×, and a compactness factor of no more than 20. In some embodiments, a lens assembly used in an imaging device described herein has (i) an overall length of from 8 mm to 12 mm, a magnification of at least 20×, and a compactness factor of no more than 17.5. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Accordingly, in some embodiments, a lens assembly used in an imaging device described herein has (i) an overall length of from 8 mm to 12 mm, a magnification of at least 40×, and a compactness factor of no more than 80. In some embodiments, a lens assembly used in an imaging device described herein has (i) an overall length of from 8 mm to 12 mm, a magnification of at least 40×, and a compactness factor of no more than 60. In some embodiments, a lens assembly used in an imaging device described herein has (i) an overall length of from 8 mm to 12 mm, a magnification of at least 40×, and a compactness factor of no more than 50. In some embodiments, a lens assembly used in an imaging device described herein has (i) an overall length of from 8 mm to 12 mm, a magnification of at least 40×, and a compactness factor of no more than 40. In some embodiments, a lens assembly used in an imaging device described herein has (i) an overall length of from 8 mm to 12 mm, a magnification of at least 40×, and a compactness factor of no more than 35. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Similarly, in some embodiments, a lens assembly used in an imaging device described herein has (i) an overall length of from 6 mm to 8 mm, a magnification of at least 4×, and a compactness factor of no more than 8. In some embodiments, a lens assembly used in an imaging device described herein has (i) an overall length of from 6 mm to 8 mm, a magnification of at least 4×, and a compactness factor of no more than 6. In some embodiments, a lens assembly used in an imaging device described herein has (i) an overall length of from 6 mm to 8 mm, a magnification of at least 4×, and a compactness factor of no more than 5. In some embodiments, a lens assembly used in an imaging device described herein has (i) an overall length of from 6 mm to 8 mm, a magnification of at least 4×, and a compactness factor of no more than 4. In some embodiments, a lens assembly used in an imaging device described herein has (i) an overall length of from 6 mm to 8 mm, a magnification of at least 4×, and a compactness factor of no more than 2. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Similarly, in some embodiments, a lens assembly used in an imaging device described herein has (i) an overall length of from 6 mm to 8 mm, a magnification of at least 8×, and a compactness factor of no more than 12. In some embodiments, a lens assembly used in an imaging device described herein has (i) an overall length of from 6 mm to 8 mm, a magnification of at least 8×, and a compactness factor of no more than 10. In some embodiments, a lens assembly used in an imaging device described herein has (i) an overall length of from 6 mm to 8 mm, a magnification of at least 8×, and a compactness factor of no more than 8. In some embodiments, a lens assembly used in an imaging device described herein has (i) an overall length of from 6 mm to 8 mm, a magnification of at least 8×, and a compactness factor of no more than 6. In some embodiments, a lens assembly used in an imaging device described herein has (i) an overall length of from 6 mm to 8 mm, a magnification of at least 8×, and a compactness factor of no more than 5. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Similarly, in some embodiments, a lens assembly used in an imaging device described herein has (i) an overall length of from 6 mm to 8 mm, a magnification of at least 10×, and a compactness factor of no more than 14. In some embodiments, a lens assembly used in an imaging device described herein has (i) an overall length of from 6 mm to 8 mm, a magnification of at least 10×, and a compactness factor of no more than 12. In some embodiments, a lens assembly used in an imaging device described herein has (i) an overall length of from 6 mm to 8 mm, a magnification of at least 10×, and a compactness factor of no more than 10. In some embodiments, a lens assembly used in an imaging device described herein has (i) an overall length of from 6 mm to 8 mm, a magnification of at least 10×, and a compactness factor of no more than 8. In some embodiments, a lens assembly used in an imaging device described herein has (i) an overall length of from 6 mm to 8 mm, a magnification of at least 10×, and a compactness factor of no more than 6. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Accordingly, in some embodiments, a lens assembly used in an imaging device described herein has (i) an overall length of from 6 mm to 8 mm, a magnification of at least 20×, and a compactness factor of no more than 25. In some embodiments, a lens assembly used in an imaging device described herein has (i) an overall length of from 6 mm to 8 mm, a magnification of at least 20×, and a compactness factor of no more than 20. In some embodiments, a lens assembly used in an imaging device described herein has (i) an overall length of from 6 mm to 8 mm, a magnification of at least 20×, and a compactness factor of no more than 17.5. In some embodiments, a lens assembly used in an imaging device described herein has (i) an overall length of from 6 mm to 8 mm, a magnification of at least 20×, and a compactness factor of no more than 15. In some embodiments, a lens assembly used in an imaging device described herein has (i) an overall length of from 6 mm to 8 mm, a magnification of at least 20×, and a compactness factor of no more than 12.5. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

Accordingly, in some embodiments, a lens assembly used in an imaging device described herein has (i) an overall length of from 6 mm to 8 mm, a magnification of at least 40×, and a compactness factor of no more than 50. In some embodiments, a lens assembly used in an imaging device described herein has (i) an overall length of from 6 mm to 8 mm, a magnification of at least 40×, and a compactness factor of no more than 40. In some embodiments, a lens assembly used in an imaging device described herein has (i) an overall length of from 6 mm to 8 mm, a magnification of at least 40×, and a compactness factor of no more than 35. In some embodiments, a lens assembly used in an imaging device described herein has (i) an overall length of from 6 mm to 8 mm, a magnification of at least 40×, and a compactness factor of no more than 30. In some embodiments, a lens assembly used in an imaging device described herein has (i) an overall length of from 6 mm to 8 mm, a magnification of at least 40×, and a compactness factor of no more than 25. In some such embodiments, the lens assembly has a Field number of no more than 15 mm. In some such embodiments, the lens assembly has a Field number of no more than 12 mm. In some such embodiments, the lens assembly has a Field number of no more than 10 mm. In some such embodiments, the lens assembly has a Field number of no more than 9 mm. In some such embodiments, the lens assembly has a Field number of no more than 8 mm. In some such embodiments, the lens assembly has a Field number of no more than 7 mm. In some such embodiments, the lens assembly has a Field number of no more than 6 mm. In some such embodiments, the lens assembly has a Field number of no more than 5 mm.

However, the present invention is not limited thereto. The image device of the present invention can have other lens assemblies. For instance, in some embodiments, the lens assembly of the present invention is configured the same as or similar to those disclosed by Blahnik, et al. “Smartphone imaging technology and its applications”, Advanced Optical Technologies, vol. 10, no. 3, 2021, pp. 145-232, which is incorporated herein for all purposes in its entirety.

In some embodiments, the lens assembly includes a compound lens having an optical axis and an image surface through which the optical axis passes. The compound lens includes a plastic lens barrel surrounding the optical axis. The plastic lens barrel includes an image-side portion and an object-side aperture through which the optical axis passes. When in use, a sample is placed at the finite focal plane of the compound lens at a distance designated as its back focal length in designs. In some embodiments, the compound lens includes two imaging lenses with two different back focal lengths. The object-side uses the imaging lens with a shorter back focal length and the image-side uses the imaging lens with a longer back focal length. The effective magnification of the whole imaging device would be determined by the ratio of the back focal lengths of the two imaging lenses. This configuration would allow the use of a variety of imaging lens designs that could result in 1×, 2×, 5×, 10×, 15×, 20× or larger magnification factors. In some embodiments, the compound lens includes between 2 and 10 lenses.

In certain embodiments, the lens assembly 120 has a short working distance. For instance, in some embodiments, a commodity (e.g., cell-phone or consumer electronics) lens is taken, reversed and used as the objective lens 121 of the lens assembly. A typical commodity lens package has more than three optical elements and a short back focal length (e.g., less than 2 mm) to focus images on the imaging sensors. It is usually less than 15 mm in thickness and 15 mm in diameter, and weighs less than 50 gram (g). A commodity lens is also inexpensive, costing a few dollars in the current market. As such, by using a reversed commodity lens as the objective lens, the microscopic imaging device of the present disclosure can achieve a short working distance, which is substantially the same as the back focal length of the commodity lens (e.g., less than 2 mm), with reduced overall size, weight and cost.

In some embodiments, The lens assembly 120 has a working distance of less than 5 mm, less than 4 mm, less than 3 mm, or less than 2 mm. In some embodiments, the working distance is less than 1.5 mm, less than 1.4 mm, less than 1.3 mm, less than 1.2 mm, less than 1.1 mm, or less than 1.0 mm. In some embodiments, the working distance is about 1.0 mm, about 1.2 mm, about 1.4 mm or about 1.6 mm.

In some embodiments, the objective lens 121 or the lens assembly 120 has a size of less than 15 mm in thickness and 15 mm in diameter. In some embodiments, the thickness of the objective lens 121 or the lens assembly 120 is less than 14 mm, 13 mm, 12 mm, 11 mm, 10 mm, 9 mm, 8 mm, 7 mm, 6 mm, 5 mm, 4 mm or smaller. In an embodiment, the objective lens 121 or the lens assembly 120 has a size of about 5 mm in thickness and about 7 mm in diameter.

In some embodiments, the objective lens 121 or the lens assembly 120 has a weight of less than 50 g, 45 g, 40 g, 35 g, 30 g, 25 g, 20 g, 15 g, 10 g, 9 g, 8 g, 7 g, 6 g, or 5 g. In an embodiment, the objective lens 121 or the lens assembly 120 has a weight of about 10 g. In another embodiment, the objective lens 121 or the lens assembly 120 has a weight of about 8 g. In still another embodiment, the objective lens 121 or the lens assembly 120 has a weight of about 6 g.

Moreover, reversing a commodity lens and using the reversed commodity lens as the objective lens allows for the use of other commodity components, resulting in further reduction in the overall size, weight and cost of the microscopic imaging device. For instance, in some embodiments, the imaging device includes an actuator 160, such as a voice coil actuator (vca) or a voice coil motor (vcm) coupled or integrated with the lens assembly to adjust a focus of the objective lens or the lens assembly. In some embodiments, the actuator 160 is a commodity autofocus vca or vcm.

In some embodiments, the actuator 160 (e.g., vca or vcm) has a size of less than 30 mm in diameter and 30 mm in thickness. In some embodiments, the diameter of the actuator is less than 32 mm, 30 mm, 28 mm, 26 mm, 24 mm, 22 mm, 20 mm or smaller. In some embodiments, the actuator is able to move or fine tune the objective lens or the lens assembly in a range of about ±1.2 mm, about ±1.1 mm, about ±1.0 mm, about ±0.9 mm, or about ±0.8 mm.

In some embodiments, the sample to be detected is loaded to a sample stage assembly 170 configured for positioning the sample chamber at the detection area. For instance, in some embodiments, the sample stage assembly is configured for moving the sample chamber with respect to the lens assembly. In some embodiments, the sample stage assembly is configured to enable a rotational field of view (FOV) selection. For instance, in some embodiments, the sample stage assembly includes a rotating spindle configured for rotating the sample chamber with respect to an optical axis of the lens assembly to enable a rotational field of view selection. In an embodiment, the sample stage assembly includes a single rotating spindle for adjusting the FOV, for instance, by allowing consistent tilting and small variation of the sample in the focal plane of the lens assembly. With a single rotating spindle, the sample stage assembly of the present disclosure has fewer moving parts than conventional sample stages that move in two axes. As a result, the imaging device of the present disclosure is less complex and less expensive. In addition, the sample stage assembly of the present disclosure enables sample preparation and processing on the same structure as imaging without requirement for additional pumps, valves, or manual intervention. In some embodiments, the sample stage assembly is configured to move the sample chamber out of the detection area and position a different sample chamber at the detection area.

The light source assembly 140 is configured to emit a light for illuminating the sample and/or exciting one or more components in the sample. For instance, in some embodiment, the light source assembly 140 is configured to emit a light including an illumination light, e.g., at least a portion of the light being an illumination light that is suitable for illuminating the sample but unable to excite any component in the sample. In some embodiments, the light source assembly 140 is configured to emit a light including an excitation light, e.g., at least a portion of the light being an excitation light that is capable of exciting one or more components in the sample. In some embodiments, the light source assembly 140 is configured to emit a light including an illumination light and an excitation light.

In some embodiments, the light source assembly 140 is configured to emit a light at one or more wavelengths or at one or more wavelength ranges. A wavelength range can be wide or narrow (e.g., wide-band or narrow-band, and the term “wavelength range” is used interchangeably with the term “wavelength band”). Different wavelength ranges can be continuous, discontinuous or overlapped. For instance, as a non-limiting example, the light source assembly 140 can be configured to emit a light at a wide and continuous wavelength range, e.g., a visible light at a wavelength range of 400 nanometers (nm)-700 nm. As another non-limiting example, the light source assembly 140 can be configured to emit a light including an excitation light at one or more excitation wavelength ranges for exciting one or more components of the sample. As a further non-limiting example, the light source assembly 140 can include a light source that emits a light at a relative wide range, and one or more filters (e.g., bandpass filter) that filter the emitted light to produce an excitation light at one or more excitation wavelength ranges for exciting one or more components of the sample. In some embodiments, the first light source assembly is configured to emit light at a single narrow wavelength band. In some embodiments, the single narrow wavelength bands has a full width at half max (FWHM) of no more than 50 nm, no more than 25 nm, no more than 10 nm, or no more than 5 nm.

The light source assembly 140 includes a light source 141. The light source 141 can include one or more laser diodes, one or more light-emitting diodes (LEDs), or any combination thereof. LEDs are more preferable than laser diodes because laser diodes may cause extra heating and photo-bleaching. In addition, laser diodes in the current market may not produce light at certain desired wavelengths or wavelength ranges for analyzing certain biological samples. In comparison, LEDs usually generate less heat and can provide more uniform light and consistent optical output power over time. In some embodiments, a single LED is able to deliver sufficient optical power to the sample plane with less heating. Accordingly, there is less opportunity for sample damage and burning.

In an embodiment, the light source 141 consists of a single LED. In some embodiments, the light source 141 includes a plurality of LEDs, e.g., 2, 3, 4, 5, 6, 7, 8, 9, 10, or more than 10 LEDs. In an embodiment, the plurality of LEDs is arranged as a single point light source. In another embodiment, the plurality of LEDs is arranged as a 2-dimensional light source, e.g., forming an array, a circle or the like on a common plane. In still another embodiment, the plurality of LEDs is arranged as a 3-dimensional light source, e.g., no common plane. In an embodiment, all of the plurality of LEDs emit a light at the same wavelength band. In another embodiment, the plurality of LEDs includes at least one LED configured to emit a light at a first narrow wavelength band and at least one LED configured to emit light at a second narrow wavelength band that is different than the first narrow wavelength band. In a further embodiment, each of the plurality of LEDs emits light at a different wavelength band.

For instance, in some embodiments, the microscopic imaging device of the present disclosure is configured for analyzing a biological sample such as a body fluid (e.g., a blood sample, a plasma sample, a buffy coat sample, a urine sample) or the like. A blood sample generally includes blood cells (e.g., red blood cells, white blood cells, and platelets) and a urine sample generally includes analytes (e.g., various types of crystals present in urine) or the like. In such embodiments, the light source 141 is configured to emit a light at one or more wavelength ranges that correspond to cell stains and/or dyes (e.g., nucleic acid, organelle, or other cell structure or sub-structure stains and/or labels that bind to cell-surface markers) or the like. In some embodiments, the light source assembly 141 is configured to emit an excitation light at one or more excitation wavelength ranges for exciting one or more components (e.g., dyes, fluorescent labels) of the sample. Upon excitation, the one or more components of the sample emits an emission light at one or more emission wavelength ranges.

In some embodiments, the light source 141 is configured to emit a light including an excitation light at one or more narrow wavelength bands. The one or more narrow wavelength bands may be selected from the group consisting of 405 nanometer (nm), 460 nm, 470 nm, 520 nm, and 638 nm. In an emblement, the light at a first narrow wavelength band excites a first component (e.g., DNA or a dye that stains DNA) in the sample and the light at a second narrow wavelength band excites a second component (e.g., RNA or a dye that stains RNA) in the sample. Upon excitation, the first component emits a light at a first emission wavelength range (e.g., green light) and the second component emits a light at a second emission wavelength range (e.g., red light). In an alternative embodiment, the light source 141 is configured to emit a light including an excitation light at a single narrow wavelength band that excites both a first component and a second component (e.g., the same dye that stains both DNA and RNA). Upon excitation, the first component emits a light at a first emission wavelength range (e.g., green light) and the second component emits a light at a second emission wavelength range (e.g., red light).

In some embodiments, the light source assembly 140 includes additional optical components. For instance, in some embodiments, the light source assembly 140 includes a collimating lens 142, a diffuser 143, a baffle 144, or any combination thereof. The additional or optional components are included to collimate the light emitted by the light source 141, soften the light, and/or prevent the light from dispersing in unwanted directions.

The detection unit 180 includes one or more detection assemblies. By way of example, FIG. 1A illustrates that the detection unit 180 includes a detection assembly 181 and a detection assembly 184. The detection assembly 181 and detection assembly 184 can be configured the same as, similar to, or differently from each other. In some embodiments, the detection assembly 181 includes a sensor 182 and a tube lens 183 that receives the light and focuses it to the sensor 182. In some embodiments, the tube lens has an effective focal length of less than 50 mm, less than 45 mm, less than 40 mm, less than 35 mm, or less than 30 mm. The use of the tube lens allows for more advanced light manipulation techniques. Similarly, in some embodiments, the detection assembly 184 includes a sensor 185 and a tube lens 186 that receives the light and focuses it to the sensor 185. In an embodiment, the sensor 185 and the tube lens 185 of the detection assembly 184 are substantially the same as the sensor 182 and the tube lens 183 of the detection assembly 181.

In some embodiments, the sensor 181 or 185 is a commodity sensor (e.g., consumer electronic camera sensor), and compatible with mobile industry processor camera serial interface standards for high speed communication between the sensor and the host processor. In some embodiments, the sensor is a charge coupled device (CCD) or the like. In some embodiments, the sensor has a cross or diagonal dimension of less than 20 mm, less than 19 mm, less than 18 mm, less than 17 mm, less than 16 mm, or less than 15 mm. In some embodiments, the sensor includes at least 500 pixels. In some embodiments, the sensor includes at least 1000 pixels, at least 2500 pixels, at least 5000 pixels, at least 10,000 pixels, at least 25,000 pixels, at least 50,000 pixels, at least 100,000 pixels, at least 250,000 pixels, at least 500,000 pixels, at least 1,000,000 pixels, at least 2,500,000 pixels, at least 5,000,000 pixels, or more pixels. Accordingly, the sensor can detect spatial responses of the sample in response to illumination and/or excitation.

In some embodiments, the detection assembly 181 and the detection assembly 184 are configured or positioned to detect a light at different wavelength ranges. For instance, in some embodiments, the imaging device or the detection unit includes a dichroic mirror 187 positioned in an optical path to receive the light from the optic assembly 130. The dichroic mirror 187 has a passband including a first portion of the one or more emission wavelength bands, and a stopband including a second portion of the one or more emission wavelength bands. In some embodiments, the dichroic mirror 187 transmits the light at one or more wavelength ranges and reflects the light at one or more different wavelength ranges. A non-limiting example is that the dichroic mirror 187 transmits a green light and reflects a red light, or vice versa. One of the detection assembly 181 and detection assembly 184 is configured or positioned to detect the light transmitted through the dichroic mirror 187, and the other of the detection assembly 181 and detection assembly 184 is configured or positioned to detect the light reflected by the dichroic mirror 187. By way of example, FIG. 1A illustrates that the detection assembly 181 detects the light transmitted through the dichroic mirror 187 and the detection assembly 184 detects the light reflected by the dichroic mirror 187. Of course, the detection assembly 181 can be configured or positioned to detect the light reflected by the dichroic mirror 187 and the detection assembly 184 can be configured or positioned to detect the light transmitted through the dichroic mirror 187.

The use of the dichroic mirror and multiple detection assemblies eliminates the requirement of a filter wheel for channel separation (e.g., separation of light at different wavelengths or wavelength bands). Moreover, it enables faster image captures with no delay for moving the filter wheel, which inherently adds time between image captures. Further, it provides a possibility for sequential or concurrent measurement of multiple channels when desired. In addition, additional dichroic mirror(s), bandpass filters and/or detection assemblies can be employed for extra channel separation.

In some embodiments, the imaging device includes other additional, optional, or alternative components. For instance, in some embodiments, the imaging device or the detection unit includes a filter 188 positioned in an optical path between the optic assembly 130 and the dichroic mirror 187 to filter out noise, any undesired light, or the like. In an embodiment, the filter 188 includes a long pass filter that reflects light at short wavelengths while transmitting or passing light at long wavelengths.

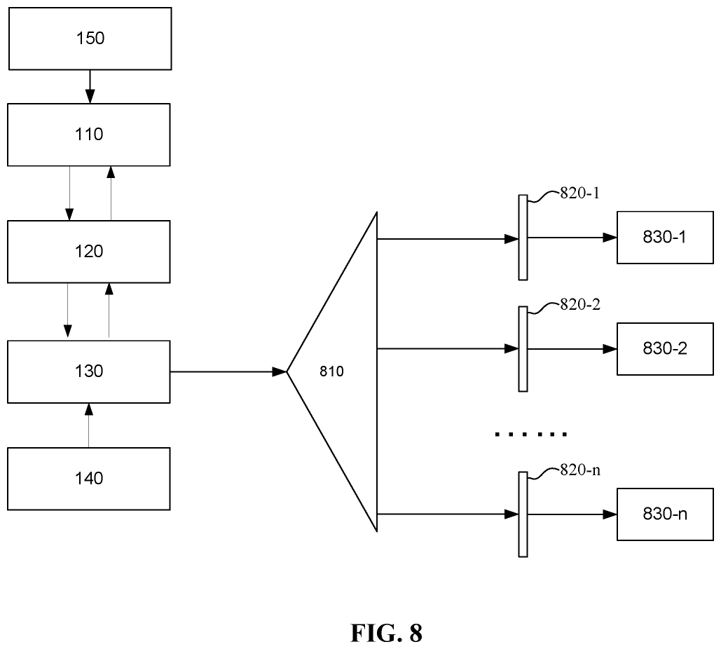

Referring to FIG. 8, in some embodiments, alternatively, optionally or additionally, the imaging device includes at least one splitter, and a plurality of filters. The one or more detection assemblies includes a plurality of detection assemblies. The at least one splitter is positioned in the second optical path between the first dichroic mirror and the plurality of detection assemblies and configured to split the one or more emission wavelength bands into a plurality of portions. Each respective filter in the plurality of filters is positioned between the at least one splitter and a corresponding detection assembly in the plurality of detection assemblies and has a respective passband in a plurality of passbands to filter a corresponding portion in the plurality of portions of the one or more emission wavelength bands. For instance, as a non-limiting example, FIG. 8 illustrates an imaging device that includes a splitter 810 and a plurality of filters 820-1, 820-2, etc. The splitter 810 is positioned in the second optical path between the dichroic mirror 131 of the optic assembly 130 and the plurality of detection assemblies 830-1, 830-2, etc. The splitter 810 is configured to split the one or more emission wavelength bands (e.g., light received from the optic assembly 130) into n portions, wherein n is an integral greater than 1. The filter 820-1 is positioned between the splitter 810 and the detection assembly 830-1 and has a first passband to filter a first portion of the one or more emission wavelength bands. The detection assembly 830-1 includes a two-dimensional optical detector (e.g., the sensor 181 or 185) configured to acquire an image of the first portion of the one or more emission wavelength bands after it passes the filter 820-1. Similarly, the filter 820-2 is positioned between the splitter 810 and the detection assembly 830-2 and has a second passband to filter a second portion of the one or more emission wavelength bands. The detection assembly 830-2 includes a two-dimensional optical detector (e.g., the sensor 181 or 185) configured to acquire an image of the second portion of the one or more emission wavelength bands after it passes the filter 820-2.

In some embodiments, the imaging device includes an additional or optional light source assembly 150. The light source assembly 150 can be configured the same as, similar to, or different than the light source assembly 140. It can be configured to emit an illumination and/or excitation light. It can also be positioned to provide an illumination and/or excitation light to the sample from the same side or different side with respect to the lens assembly. For instance, in some embodiments, the sample is positioned with a first side (e.g., the lower side of the sample in the figure) facing the lens assembly. The light source assembly 150 can be positioned to provide an illumination and/or excitation light that arrives at the first side of the sample perpendicularly or at an angle. The light source assembly 150 can also be positioned to provide an illumination and/or excitation light that arrives at a second side (e.g., the upper side of the sample in the figure) of the sample perpendicularly or at an angle. By way of example, the light source assembly 150 is illustrated to provide illumination and/or excitation light from a different side with respect to the lens assembly.

The use of the light source assembly 140 and light source assembly 150 enables epifluorescence fluorescence imaging technique in combination with other imaging modalities such as side-scatter, darkfield, and/or brightfield imaging techniques. For instance, in some embodiments, the light source assembly 140 is configured to provide a light including an excitation light to excite one or more components of the sample, which emit a fluorescent light at one or more emission wavelengths or wavelength ranges upon excitation. The light source assembly 150 includes one or more of a brightfield light source 152, a darkfield light source 153, and a side scatter light source 151. The light source assembly 150 emits a light including an illumination light that propagates to a different side of the sample with respect to the lens assembly. The sample reflects, scatters, absorbs and/or transmits the illumination light from the light source assembly 150. The lens assembly collects at least a portion of the light emitted, scattered and/or transmitted by the sample and passes the collected light for detection by the detection unit 180.

The brightfield light source is generally positioned such that it illuminates the sample at an angle of between about 45° and about 90° relative to an illuminated area of the sample or relative to the optical axis of the lens assembly. The darkfield light source is generally positioned such that it illuminates the sample at an angle of between about 0° and about 45° relative to an illuminated area of the sample or relative to the optical axis of the lens assembly. In some embodiments, the light source assembly 150 includes other optional or additional components such as a diffuser 154 and one or more apertures 155. In some embodiments, the diffuser 154 is configured to soften or spread the light produced by the brightfield light source, darkfield light source or side scatter light source. In some embodiments, the one or more apertures 155 are configured to allow the light produced by the brightfield light source, darkfield light source or side scatter light source to pass through.

In some embodiments, the brightfield light source 152 includes one or more LEDs, e.g., 1, 2, 3, 4, 5, 6, 7, 8, 9, 10 or more LEDs. In some embodiments, the darkfield light source 153 includes one or more LEDs, e.g., 1, 2, 3, 4, 5, 6, 7, 8, 9, 10 or more LEDs. In some embodiments, the side scatter light source 151 includes one or more LEDs, e.g., 1, 2, 3, 4, 5, 6, 7, 8, 9, 10 or more LEDs. In an embodiment, each of the brightfield, darkfield and scatter light sources includes one or more LEDs.

While FIG. 1A illustrates the microscopic imaging device including two light source assemblies and two detection assemblies, it should be noted that a microscopic imaging device of the present disclosure can include one, two, three, four or more light source assemblies for illuminating and/or exciting the sample and one, two, three, four or more detection assemblies for detecting the light from the sample (e.g., light emitted by, reflected by, scattered by, transmitted through the sample, or any combination thereof). In various exemplary embodiments, the lens assembly, at least one light source assembly, and at least one detection assembly are positioned to form an epi-configuration, i.e., both the light from the at least one light source assembly and the light from the sample to be detected by the at least one detection assembly travel through the same objective lens of the lens assembly.

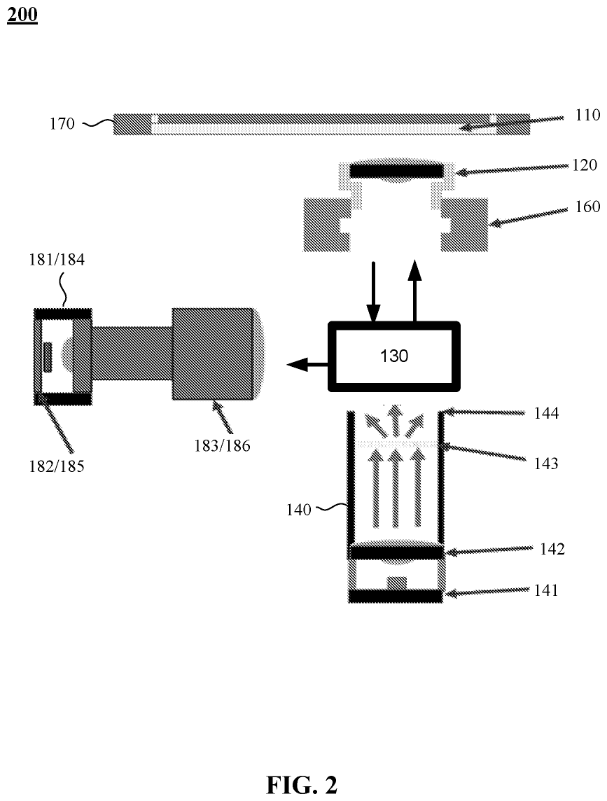

For instance, as a non-limiting example, FIG. 2 illustrates an exemplary microscopic imaging device 200 including a single light source assembly (e.g., the light source assembly 140) and a single detection assembly (e.g., the detection assembly 181 or 184) in accordance with some exemplary embodiments of the present disclosure. In the illustrated embodiment, the optic assembly 130 relays the light emitted by the single light source assembly to the lens assembly 120 and relays the light from the sample collected and passed by the lens assembly 120 to the single detection assembly.

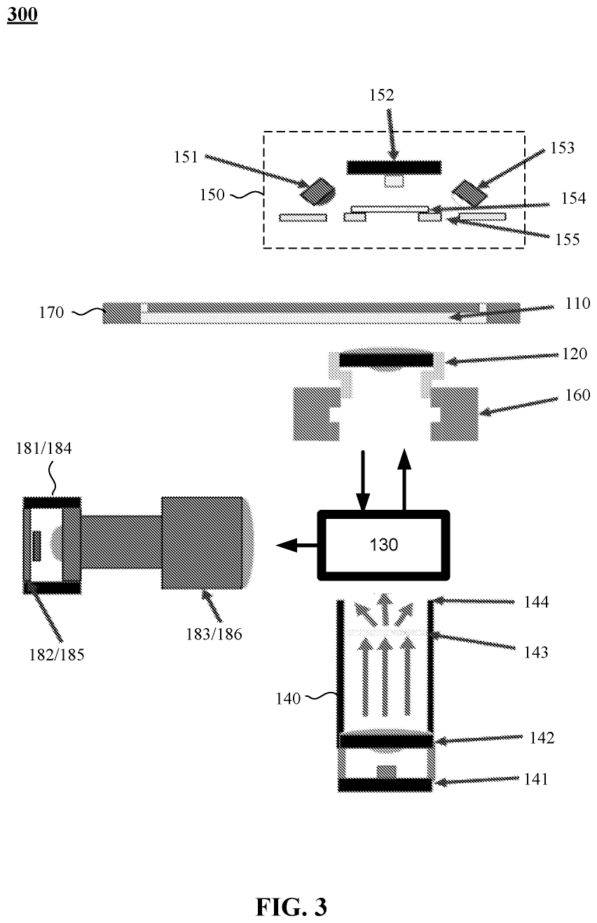

As another non-limiting example, FIG. 3 illustrates an exemplary microscopic imaging device 300 including two light source assemblies (e.g., the light source assembly 140 and the additional or optional light source assembly 150) and a single detection assembly (e.g., the detection assembly 181 or 184) in accordance with some exemplary embodiments of the present disclosure. The imaging device 300 is similar to the imaging device 200 except the presence of the additional or optional light source assembly. In some embodiments, the light source assembly 140 provides an excitation light to the sample, and the sample produces an emission light in response to the excitation light. The light source assembly 150 produces an illumination light to the sample, and the sample reflects, scatters, absorbs and/or transmits the light from the light source assembly 150. The lens assembly collects at least a portion of the light emitted, scattered and/or transmitted by the sample and passes the collected light to the optic assembly 130. The optic assembly 130 relays the light from the sample collected and passed by the lens assembly 120 to the single detection assembly.

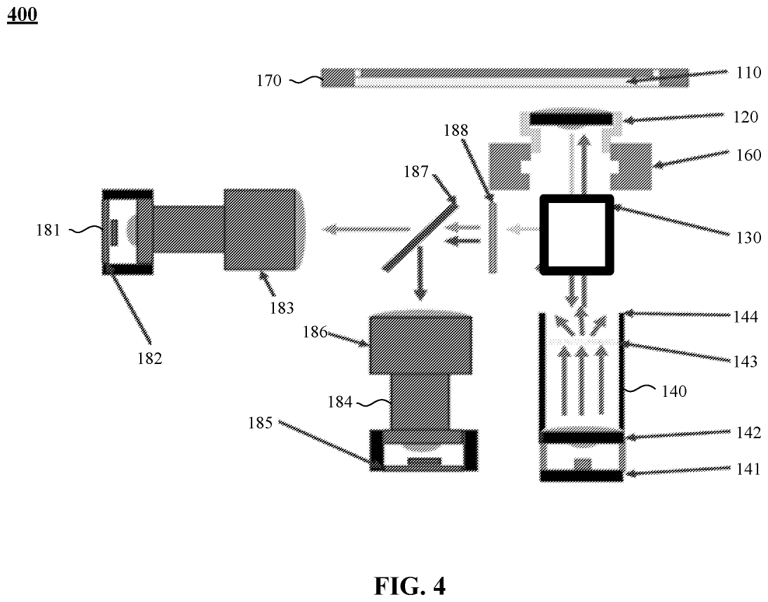

As a further non-limiting example, FIG. 4 illustrates an exemplary microscopic imaging device 400 including a single light source assembly (e.g., the light source assembly 140) and two detection assemblies (e.g., the detection assembly 181 and detection assembly 184) in accordance with some exemplary embodiments of the present disclosure. The imaging device 400 is similar to the imaging device 100 except the absence of the additional or optional light source assembly. In some embodiments, the light source assembly 140 is configured to provide an excitation light to the sample, and the sample produces an emission light in response to the excitation light. The lens assembly collects at least a portion of light emitted by the sample and passes the collected light to the optic assembly 130 for detection by the detection assemblies. In some embodiments, the light source assembly 140 is configured to provide an illumination light to the sample, and the sample reflects, scatters, absorbs and/or transmits the light from the light source assembly 140. The lens assembly collects at least a portion of light scattered and/or reflected by the sample and passes the collected light to the optic assembly 130 for detection by the detection assemblies. In some embodiments, the light source assembly 140 is configured to provide both an excitation light and an illumination light to the sample, and the lens assembly collects at least a portion of light emitted, scattered and/or reflected by the sample the sample and passes the collected light to the optic assembly 130 for detection by the detection assemblies.

A portable microscopic imaging device (e.g., the imaging device 100, 200, 300 or 400) of the present disclosure is generally small, having dimensions of less than 25 centimeters (cm) in length, less than 25 cm in width, and 25 cm in height. In some embodiments, a portable microscopic imaging device (e.g., the imaging device 100, 200, 300 or 400) of the present disclosure is about 10 times, about 12 times, about 14 times, about 16 times, about 18 times, about 20 times, about 22 times, about 24 times, about 26 times, about 28 time or about 30 times smaller than some existing microscopic imaging devices. In some embodiments, a portable microscopic imaging device of the present disclosure is more than 30 times smaller than some existing microscopic imaging devices. For instance, in some embodiments, a portable microscopic imaging device of the present disclosure has an overall size that is smaller than about 200 mm in length, about 25 mm in width and about 35 mm in height. In some embodiments, a portable microscopic imaging device of the present disclosure has an overall size that is smaller than about 200 mm in length, about 22 mm in width and about 33 mm in height. In some embodiments, a portable microscopic imaging device of the present disclosure has an overall size that is smaller than about 180 mm in length, about 20 mm in width and about 30 mm in height. In some embodiments, the overall length of a portable microscopic imaging device of the present disclosure is less than 200 mm, less than 190 mm, less than 180 mm, less than 170 mm, less than 160 mm, less than 150 mm, less than 140 mm, less than 130 mm, less than 120 mm, or smaller. In some embodiments, the overall width of a portable microscopic imaging device of the present disclosure is less than 22 mm, less than 21 mm, less than 20 mm, less than 19 mm, less than 18 mm, less than 17 mm, less than 16 mm, less than 15 mm, or smaller. In some embodiments, the overall height of a portable microscopic imaging device of the present disclosure is less than 35 mm, less than 34 mm, less than 33 mm, less than 32 mm, less than 31 mm, less than 30 mm, less than 29 mm, less than 28 mm, less than 27 mm, less than 26 mm, less than 25 mm, or smaller. In some embodiments, the portable microscopic imaging device of the present disclosure weighs less than 300 grams (g), less than 280 g, less than 260 g, less than 240 g, less than 220 g, less than 200 g, less than 180 g, less than 160 g, less than 150 g, or lighter.

In some embodiments, a portable microscopic imaging device of the present disclosure has a f-number of about f/2.0, about f/2.1, about f/2.2, about f/2.3, about f/2.4, or about f/2.5. In some embodiments, a portable microscopic imaging device of the present disclosure has a f-number of greater than about f/2.0, greater than about f/2.5, greater than about f/3.0, greater than about f/3.5, greater than about f/4.0, greater than about f/4.5, greater than about f/5.0, or higher. In some embodiments, a portable microscopic imaging device of the present disclosure has a magnification of less than about 10×. In some embodiments, a portable microscopic imaging device of the present disclosure has a magnification of less than about 5×, 10×, 15×, 20×, 25×, 30×, 40×, 50×, 75×, 100×, or greater.

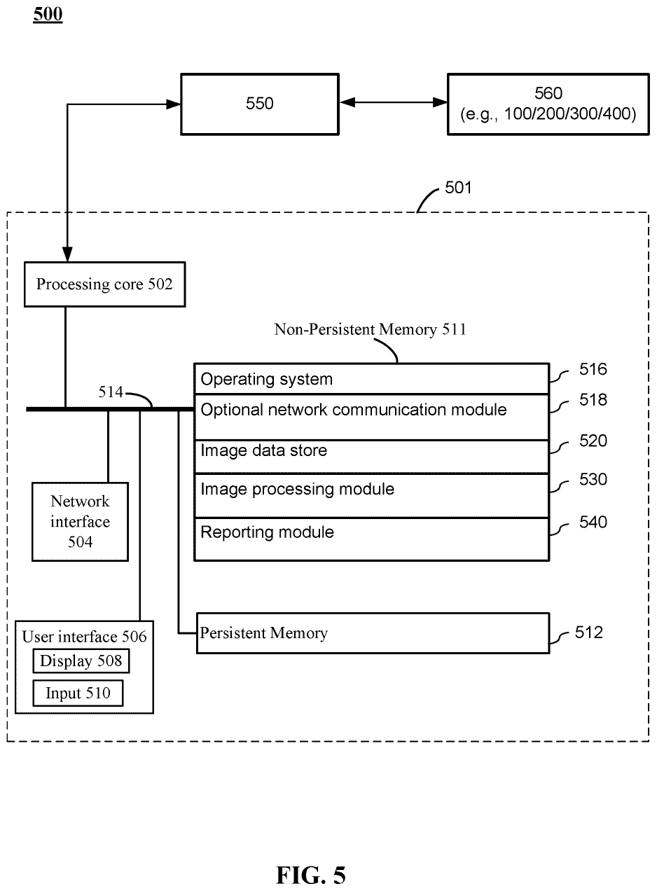

Referring now to FIG. 5, there is shown a block diagram illustrating a system 500 for analyzing biological samples in accordance with some embodiments of the present disclosure. The system 500 includes an imaging device 560, a control unit 550, and a computing device 501. The imaging device 560 can be any of the imaging devices 100, 200, 300 and 400 disclosed herein or the like.

The control unit can be a standalone unit. The control unit can also be embedded or integrated with the imaging device 560 or computing device 501. In some embodiments, the control unit 550 is configured to control the imaging device 560 so that detection (e.g., capture data or images) can be performed in a desired or defined manner. For instance, in embodiments where the imaging device includes the light source assembly 140, the control unit is configured to selectively activate the light source assembly 140 or any individual light source (e.g., one or more LEDs) in the light source assembly 140. In embodiments where the imaging device includes the light source assembly 150, the control unit is configured to selectively activate the light source assembly 150 or any individual light source in the light source assembly 150 (e.g., the brightfield/darkfield/side scatter light source or one or more LEDs of the brightfield/darkfield/side scatter light source). In embodiments where the imaging device includes the light source assembly 140 and light source assembly 150, the control unit is configured to selectively activate the light source assembly, the second light source assembly, any individual light source in the first light source assembly, any individual light source in the second light source assembly, or any combination thereof in defined sequences. In some embodiments, the control unit is configured to control the autofocus vca or vcm, one or more light source assemblies, and one or more detection assemblies to perform the whole data or image capture procedure in a defined manner (e.g., sequential, concurrent, or sequential with defined sequences).

In some embodiments, the computing device 501 is configured the same as or similar to the computing device disclosed in U.S. patent application Ser. No. 17/371,746, the content of which is hereby incorporated by reference in its entirety. For instance, in some embodiments, the computing device 501 includes a network interface 504. In some embodiments, the network interface 504 interconnects components within the computing device 501, as well as optional external systems and devices, through one or more communication networks (e.g., through optional network communication module 518). In some embodiments, the network interface 504 optionally provides communication via the Internet, one or more local area networks (LANs), one or more wide area networks (WANs), other types of networks, or a combination of such networks.

Examples of networks include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

The computing device 501 in some embodiments includes one or more processing units (CPU(s)) 502 (e.g., a processor, a processing core, etc.), one or more network interfaces 504, a user interface 506 including (optionally) a display 508 and an input system 510 (e.g., an input/output interface, a keyboard, a mouse, etc.) for use by the user, memory (e.g., non-persistent memory 511, persistent memory 512), and one or more communication buses 514 for interconnecting the aforementioned components. The one or more communication buses 514 optionally include circuitry (sometimes called a chipset) that interconnects and controls communications between system components. The non-persistent memory 511 typically includes high-speed random access memory, such as DRAM, SRAM, DDR RAM, ROM, EEPROM, flash memory, whereas the persistent memory 512 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 512 optionally includes one or more storage devices remotely located from the CPU(s) 502. The persistent memory 512, and the non-volatile memory device(s) within the non-persistent memory 512, include non-transitory computer readable storage medium. In some embodiments, the non-persistent memory 511 or alternatively the non-transitory computer readable storage medium stores the following programs, modules and data structures, or a subset thereof, sometimes in conjunction with the persistent memory 512:
an operating system 516 (e.g., ANDROID, IOS, DARWIN, RTXC, LINUX, UNIX, OS X, WINDOWS, or an embedded operating system such as VxWorks), which includes procedures for handling various basic system services and for performing hardware dependent tasks;
an optional network communication module (or instructions) 518 for connecting the computing device 501 with other devices and/or a communication network;
an image data store 520 for storing pixelated images, e.g., digital images, for one or more samples;
an image processing module 530 including one or more algorithms or models for analyzing the data or images; and
optional reporting module 540 for generating a report on the analysis (e.g., on cell composition for a biological sample).

In some embodiments, the image processing module 530 includes one or more algorithms or models such as those disclosed in U.S. patent application Ser. No. 17/371,746, the content of which is hereby incorporated by reference in its entirety. Examples of algorithms or models include, but are not limited to, an identification algorithm for identifying pixel sets in images corresponding to individual components (e.g., cells) present in a biological sample; a feature extraction algorithm for determining imaging features for individual components (e.g., cells) present in a biological sample based on pixel values for pixel sets identified; one or more differentiation models for differentiating between types (e.g., different types of blood cells, such as red blood cells, white blood cells, and platelets) based on imaging features for individual components present in a biological sample; and cell counting algorithm for obtaining an aggregate count for each cell type present in a biological sample.

In some embodiments, the image processing module 530 also includes one or more mixers/unmixers, e.g., algorithms configured to replicate and mix first signals and second signals to generate a set of spectro-spatial responses. In some embodiments, the first signals and the second signals are representative of the responses of the sample to a light at a first wavelength or wavelength range and a light at a second wavelength or wavelength range, respectively. In some embodiments, the one or more one or more mixers/unmixers include a non-transitory computer readable medium storing instructions that, when executed by a processor, perform a machine learning algorithm to count, size, and/or speciate one or more cell types in the sample.

In various embodiments, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 511 optionally stores a subset of the modules and data structures identified above. Furthermore, in some embodiments, the memory stores additional modules and data structures not described above. In some embodiments, one or more of the above identified elements is stored in a computing device, other than that of the computing device 501, that is addressable by the computing device 501 so that the computing device 501 may retrieve all or a portion of such data when needed.

While the computing device 501 is illustrated as a computer system, other topologies of the computing device 501 are possible. For instance, in some embodiments, the computing device 501 can in fact constitute several computer systems that are linked together in a network, or be a virtual machine or a container in a cloud computing environment. As such, the exemplary topology shown in FIG. 5 merely serves to describe the features of an embodiment of the present disclosure in a manner that will be readily understood to one of skill in the art.

In some embodiments, one or more components of the computing device 501 may be embedded or integrated with the imaging device. For instance, in an embodiment, a processor is integrated with the imaging device for processing the light detected or images captured by one or more detection assemblies of the imaging device.

In some embodiments, the system 500 is configured to analyze a biological sample, such as a blood sample, a plasma sample, a buffy coat sample, a urine sample, or the like. In some embodiments, the biological sample contains cells of one or more types, e.g., a sample of blood cells containing red blood cells (RBCs), white blood cells (WBCs), and platelets. In some embodiments, the biological sample includes analytes present in urine, e.g., various types of crystals present in urine. In some embodiments, the sample is processed and tagged with fluorescent markers of interest, such as cell surface antigens, DNA/RNA stains or the like. The sample is then loaded onto the imaging device that captures one or more data sets of spectral, spatial and/or temporal responses from the sample in response to the illumination and/or excitation. The data is analyzed, e.g., at the computing device, to provide information about the sample. In some embodiments, the system is configured to identify the total number of cells within the analyte as well as their species, such as red blood cells (RBCs), platelets, white blood cells (WBCs) and their different types. In some embodiments, a combination of spatial, spectral, and/or temporal information about these cells allowed for the identification, counting and speciation of different types of cells within the target specimen in a fast and reliable manner. In some embodiments, the system is configured to provide identification of the types of crystals present in a urine sample.

In some embodiments, the one or more light source assemblies of the imaging device 560 emit light at a wavelength independently selected from the group consisting of 405 nm, 460 nm, 470 nm, 520 nm, and 638 nm. In some embodiments, the one or more light source assemblies include respective light sources that are configured to emit a light at one of 405 nm, 460 nm, 470 nm, 520 nm, and 638 nm. For instance, in some embodiments, the light source assembly 140 includes at least one LED to emit a light at 460 nm and at least one LED to emit light at 470 nm for exciting the DNA/RNA stains. In some embodiments, the light source assembly 140 includes one or more LEDS to emit a light at a single wavelength or a single wavelength range for exciting the DNA/RNA stains.

In some embodiments, the imaging device 560 is configured to acquire different types of images. For instance, in some embodiments, the imaging device is configured to acquire fluorescence emission from the sample (e.g., fluorescence emission from analytes such as blood cells or urine crystals in the sample), backscatter from the sample (e.g., backscatter from analytes such as blood cells or urine crystals in the sample), transmission through the sample (e.g., transmission through analytes such as blood cells or urine crystals in the sample, from which absorption and/or attenuation values can be derived), bright field imaging of the sample, etc. In some embodiments, absorption values derived from transmission images are used to determine analyte volumes (e.g., cell volumes or urine crystal volumes).

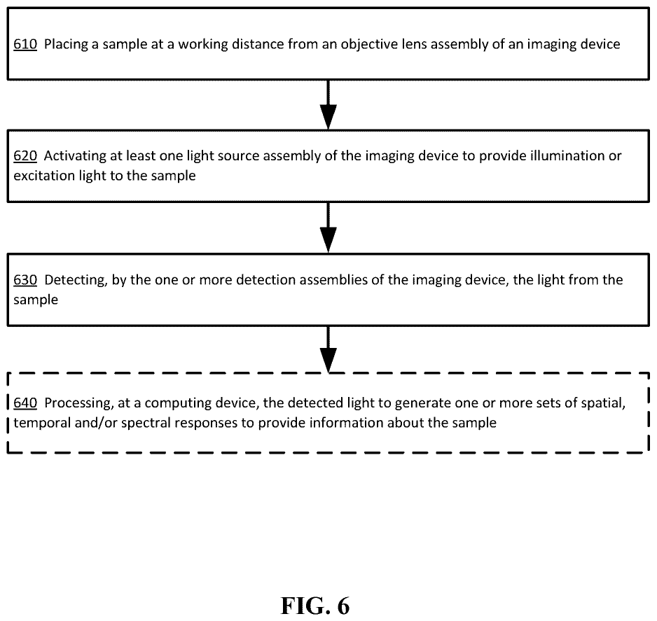

Referring to FIG. 6, there is shown a flowchart illustrating an exemplary method 600 for imaging and/or analyzing a biological sample in accordance with some embodiments of the present disclosure. In the flowchart, the preferred parts of the method are shown in solid line boxes, whereas additional, optional, or alternative parts of the method are shown in dashed line boxes. It should be noted that the processes disclosed herein and exemplified in the flowchart can be, but do not have to be, executed in full or in the order as they are presented.

The method 600 includes placing a sample at a working distance from a lens assembly of an imaging device (e.g., the imaging device 100, 200, 300 or 400) at step 610. In some embodiments, the sample is a blood sample, a plasma sample, a buffy coat sample, a urine sample, or the like. The lens assembly of the imaging device has a short working distance of less than 5 mm, less than 4 mm, less than 3 mm, or less than 2 mm. In addition to the lens assembly, the imaging device includes one or more light source assemblies and one or more detections assemblies. The lens assembly, at least one light source assembly, and at least one detection assembly are positioned to form an epi-configuration. In some embodiments, placing a sample at the working distance includes adjusting the focus and/or FOV of the imaging device. In some embodiments, the focus and/or FOV of the imaging device are adjusted automatically once the sample is placed at the working distance from the lens assembly of an imaging device.

The method 600 also includes activating at least one light source assembly of the imaging device to provide an illumination and/or excitation light to the sample at step 620. In an embodiment, the method activates one light source assembly or an individual light source of a light source assembly at a time. In another embodiment, the method simultaneously activates two or more light source assemblies or two or more individual light sources. In some embodiments, when activated, a light source assembly emits a light including an excitation light at one or more excitation wavelength ranges for exciting one or more components of the sample. For instance, in some embodiments, an activated light source assembly emits a light including an excitation light of certain wavelength or wavelengths for exciting the DNA/RNA stains. Upon excitation, the one or more components of the sample emit an emission light at one or more emission wavelength ranges. For instance, in some embodiments, the DNA/RNA stains emit a green light and a red light upon excitation.

The method 600 further includes detecting, by the one or more detection assemblies of the imaging device, the light from the sample. Depending on the configuration of the imaging device (e.g., positions of the one or more light source assemblies relative to the sample, the spectrum of the lights from the one or more light source assemblies), procedure of the detection (e.g., which light source assembly is activated) and/or other factors, the light out of the sample can include the light emitted by the sample, scattered by the sample, reflected by the sample, transmitted through the sample, or any combination thereof. Depending on the configuration of the imaging device (e.g., the presence or absence of other optics such as filters, splitter, absorbers or the like) and/or other factors, the light arriving at the one or more detection assemblies can be substantially the same as or different than the light out of the sample. In an embodiment, the light arriving at the one or more detection assemblies is an emission light (e.g., fluorescence emission) at a single wavelength or a single wavelength range. In another embodiment, the light arriving at the one or more detection assemblies is an emission light of multiple (e.g., 2, 3, 4, or more) wavelengths or wavelength ranges. In still another embodiment, the light arriving at the one or more detection assemblies is a light scattered by the sample, reflected by the sample and/or transmitted through the sample. In yet another embodiment, the light arriving at the one or more detection assemblies is a combination of an emission light and a light scattered by the sample, reflected by the sample and/or transmitted through the sample.

The one or more detection assemblies detect the light from the sample (e.g., capture an image of an area of the sample). In some embodiments, additional images are captured at different wavelengths by providing an illimitation and/or excitation light at different wavelengths to the biological sample and detecting the light from the sample in response to the illimitation and/or excitation light of different wavelengths. In some embodiments, the illumination and/or excitation light at different wavelengths is provided to the sample sequentially, and detection of the light from the sample is conducted sequentially. Alternatively, in some embodiments, the illumination and/or excitation light at different wavelengths is provided to the sample concurrently and detection of the light from the sample is conducted concurrently.

In some embodiments, the method 600 also includes processing, e.g., at the computing device 501, the detected light (e.g., captured images) to generate one or more sets of spatial, temporal and/or spectral responses to provide information about the sample. For instance, in an embodiment, the one or more detection assemblies receive the light emitted from the sample. The method processes the detected emission light to generate one or more sets of spectral and/or spatial responses, each set representing a spatial distribution of a corresponding component (e.g., a specific type of cell) in the one or more components over the area of the sample.

In some embodiments, the biological sample is a sample of blood cells containing red blood cells (RBCs), white blood cells (WBCs), and platelets. The method processes the detected light (e.g., captured images) to identify the RBCs, WBCs, platelets and their different types, and/or to provide a complete blood count. In some embodiments, the biological sample includes analytes present in urine, e.g., various types of crystals present in urine. The method 600 processes the detected light to identify the types of crystals present in the urine sample.

### TERMINOLOGIES AND REFERENCES CITED

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will be understood that the terms “left” or “right”, “top” or “bottom”, “lower” or “upper”, “interior” or “exterior”, and etc. are used to describe features of the exemplary embodiments with reference to the positions of such features as displayed in the figures. It will be understood that, although the terms “first,” “second,” etc. may be used herein to describe various elements, these elements should not be limited by these terms. These terms are only used to distinguish one element from another. For example, a first element could be termed a second element, and, similarly, a second element could be termed a first element, without changing the meaning of the description, so long as the “first element” and the “second element” are renamed consistently.

As used herein, the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to +5%.

All references cited herein are incorporated herein by reference in their entirety and for all purposes to the same extent as if each individual publication or patent or patent application was specifically and individually indicated to be incorporated by reference in its entirety for all purposes.

What is claimed is:

1. An imaging device, comprising:
a lens assembly comprising a compound lens configured to be in optical communication with a sample chamber at a detection area, wherein the lens assembly has (i) an optical path of less than about 15 mm, (ii) a working distance of no more than about 5 mm, (iii) a weight of no more than about 50 grams, or any combination thereof;
a first light source assembly comprising a first light source configured to emit light onto the sample chamber at the detection area, wherein the first light source assembly is positioned on the same side of the sample chamber as the lens assembly; and
one or more detection assemblies in optical communication with the sample chamber at the detection area, wherein the one or more detection assemblies are configured to detect light traveling from the sample chamber at the detection area through the lens assembly;
wherein the imaging device is configured to define a first optical path in which light emitted from the first light source passes through the lens assembly and is directed onto the sample chamber at the detection area, and
wherein the imaging device is configured to define a second optical path in which light returning from the sample chamber at the detection area on the sample stage assembly passes through the lens assembly and at least a portion of the light returning from the sample chamber at the detection area is directed to the one or more detection assemblies.

2. The imaging device of claim 1, wherein the lens assembly has more than three optical elements.

3. The imaging device of claim 1 or 2, wherein:
a first optical element in the more than three optical elements that is closest to an object side plane of the lens assembly has a first clear aperture diameter and is at a first distance relative to the object side plane of the lens assembly; and
a second optical element in the more than three optical elements that is farthest to the object side plane of the lens assembly has a second clear aperture diameter and is at a second distance relative to the object side plane of the lens assembly,
wherein the first clear aperture diameter is equal to or greater than the second clear aperture diameter, and the first distance is equal to or greater than 0.4 times of the second distance.

4. The imaging device of claim 3, wherein the first distance defines the working distance of the lens assembly.

5. The imaging device of claim 3 or 4, wherein the first optical element in the more than three optical elements has a variable optical power across a radial direction from an optical axis of the first optical element or of the lens assembly.

6. The imaging device of claim 5, wherein the variable optical power of the first optical element allows for a wider field of view, reduces aberration along the optical axis, or both.

7. The imaging device of any one of the preceding claims, further comprising a sample stage assembly configured for positioning the sample chamber at the detection area.

8. The imaging device of claim 7, wherein the sample stage assembly is configured for moving the sample chamber with respect to the lens assembly.

9. The imaging device of claim 7 or claim 8, wherein the sample stage assembly comprises a rotating spindle configured for rotating the sample chamber with respect to an optical axis of the lens assembly to enable a rotational field of view selection.

10. The imaging device of any one of claims 7-9, wherein the sample stage assembly is configured to move the sample chamber out of the detection area and position a different sample chamber at the detection area.

11. The imaging device of any one of the preceding claims, wherein the lens assembly has a back focal plane of no more than 8 mm, no more than 7.5 mm, no more than 7 mm, no more than 6.5 mm, no more than 6 mm, no more than 5.5, or no more than 5 mm.

12. The imaging device of any one of the preceding claims, wherein the compound lens comprises a plurality of lenses packaged in a plastic lens barrel.

13. The imaging device of claim 12, wherein:
the plastic lens barrel surrounds an optical axis of the compound lens and has an object-side and an image-side; and
the compound lens comprises a first imaging lens with a relatively shorter back focal length at the object-side and a second imaging lens with a relatively longer back focal length.

14. The imaging device of claim 13, wherein the compound lens produces 1×, 2×, 5×, 10×, 15×, 20× or larger magnification.

15. The imaging device of any one of the preceding claims, wherein the compound lens comprises between 2 and 10 lenses.

16. The imaging device of any one of the preceding claims, further comprising a first dichroic mirror, wherein the first dichroic mirror:
(i) has a passband comprising either (a) an excitation wavelength band emitted by the first light source, or (b) one or more emission wavelength bands;
(ii) has a stopband comprising the other of (a) the excitation wavelength band emitted by the first light source, or (b) the one or more emission wavelength bands;
(iii) is positioned in the first optical path between the lens assembly and the first light source assembly; and
(iv) is positioned in the second optical path between the lens assembly and the one or more detection assemblies.

17. The imaging device of claim 16, further comprising a second dichroic mirror positioned in the second optical path between the first dichroic mirror and the one or more detection assemblies, wherein:
(i) the second dichroic mirror has a passband comprising a first portion of the one or more emission wavelength bands;
(ii) the second dichroic mirror has a stopband comprising a second portion of the one or more emission wavelength bands;
(iii) the one or more detection assemblies comprise a first detection assembly comprising a first two-dimensional optical detector configured to acquire an image at a waveband in the first portion of the one or more emission wavelength bands; and
(iv) the one or more detection assemblies comprise a second detection assembly comprising a second two-dimensional optical detector configured to acquire an image at a waveband in the second portion of the one or more emission wavelength bands.

18. The imaging device of claim 17, wherein:
the first detection assembly comprises a first tube lens positioned between the second dichroic mirror and the first two-dimensional optical detector, and
the second detection assembly comprises a second tube lens positioned between the second dichroic mirror and the second two-dimensional optical detector.

19. The imaging device of claim 16, further comprising:
at least one splitter; and
a plurality of filters,
wherein:
the one or more detection assemblies comprises a plurality of detection assemblies;
the at least one splitter is positioned in the second optical path between the first dichroic mirror and the plurality of detection assemblies and configured to split the one or more emission wavelength bands into a plurality of portions;
each respective filter in the plurality of filters is positioned between the at least one splitter and a corresponding detection assembly in the plurality of detection assemblies and has a respective passband in a plurality of passbands to filter a corresponding portion in the plurality of portions of the one or more emission wavelength bands; and
the corresponding detection assembly in the plurality of detection assemblies comprises a two-dimensional optical detector configured to acquire an image of the corresponding portion in the plurality of portions of the one or more emission wavelength bands after it passes the respective filter in the plurality of filters.

20. The imaging device of any one of the preceding claims, further comprising a voice coil actuator, coupled or integrated with the lens assembly, that is configured to adjust a focus of the compound lens.

21. The imaging device of claim 20, wherein the voice coil actuator is smaller than 20 mm in width, 20 mm in length, and 20 mm in height.

22. The imaging device of claim 20, wherein the focus of the compound lens of the lens assembly is adjustable in a range of no more than about ±2 mm, about ±1.5 mm, about ±1.0 mm, or about ±0.75 mm.

23. The imaging device of any one of the preceding claims, further comprising a second light source assembly, wherein:
the second light source assembly is positioned on the opposite side of the sample stage assembly as the lens assembly,
the second light source assembly is configured to emit light onto the sample chamber at the detection area, and
the imaging device is configured to define a third optical path in which light emitted from the second light source assembly passes through the sample chamber at the detection area and the lens assembly, and at least a portion of the light that passed through the sample chamber at the detection area is directed to the one or more detection assemblies.

24. The imaging device of claim 23, wherein the second light source assembly comprises a brightfield light source, a darkfield light source, a side scatter light source, or any combination thereof.

25. The imaging device of claim 24, wherein the brightfield light source illuminates the sample chamber at an angle of between about 45° and about 90° relative to an illuminated area of the sample.

26. The imaging device of claim 24 or 25, wherein the darkfield light source illuminates the sample chamber at an angle of between about 0° and about 45° relative to an illuminated area of the sample.

27. The imaging device of any one of claims 24-26, wherein the second light source assembly further comprises a diffuser to soften or spread light produced by the brightfield light source, darkfield light source, or side scatter light source.

28. The imaging device of any one of claims 23-27, wherein a control unit is configured to selectively activate the first light source assembly, the second light source assembly, any individual light source in the first light source assembly, any individual light source in the second light source assembly, or any combination thereof.

29. The imaging device of any one of the preceding claims, wherein the sample chamber is a portion of a centrifugal micro-fluidic biodisk, a portion of a capillary tube device, or a portion of a flow cell device.

30. The imaging device of claim 29, wherein the centrifugal micro-fluidic biodisk comprises an elongated reservoir positioned radially about a rotational axis of the centrifugal micro-fluidic biodisk at a first distance from the rotational axis.

31. The imaging device of any one of the preceding claims, wherein the compound lens is a commodity lens reversely positioned such that the sample chamber is located at a back focal plane of the commodity lens.

32. The imaging device of any one of the preceding claims, wherein the imaging device has dimensions of less than 25 centimeters (cm) in length, less than 25 cm in width, and 25 cm in height.

33. The imaging device of any one of the preceding claims, wherein the first light source assembly is configured to emit light at a single narrow wavelength band.

34. The imaging device of claim 33, wherein the single narrow wavelength bands has a full width at half max (FWHM) of no more than 50 nm, no more than 25 nm, no more than 10 nm, or no more than 5 nm.

35. The imaging device of any one of the preceding claims, wherein the first light source assembly comprises one or more light-emitting diodes (LEDs).

36. The imaging device of claim 35, wherein the one or more LEDs comprises a first LED configured to emit light at a first narrow wavelength band and a second LED configured to emit light at a second narrow wavelength band.

37. The imaging device of any one of the preceding claims, wherein the first light source is a single LED.

38. The imaging device of any one of the preceding claims, wherein the lens assembly has an overall length that is about 5 mm to about 20 mm, and a diagonal dimension at an object side plane of the lens assembly that is about 3 mm to 20 mm.

39. The imaging device of claim 38, wherein the overall length is about 10 mm, and the diagonal dimension is about 5.0 mm, about 5.8 mm, about 7.0 mm, about 8.0 mm, about 9.0 mm, or about 12.0 mm.

40. The imaging device of claim 38, wherein the overall length is about 7 mm, and the diagonal dimension is about 5.0 mm, about 5.8 mm, about 7.0 mm, about 8.0 mm, about 9.0 mm, or about 12.0 mm.

41. A system, comprising:
the imaging device of any one of the preceding claims configured to capture one or more images of a sample in the sample chamber at the detection area;
a control unit in wire or wireless communication with the imaging device and configured to control the imaging device; and
a computing device in wire or wireless communication with the control unit to process the one or more captured images of the sample.

42. The system of claim 41, wherein the control unit is a standalone unit.

43. The system of claim 41, wherein the control unit is embedded or integrated with the imaging device.

44. The system of claim 41, wherein the control unit is embedded or integrated with the computing device.

45. A method, comprising:
placing the sample chamber containing a sample at the working distance from the lens assembly of the imaging device of any one of claims 1-40;
activating the first light source assembly to provide an excitation light to the sample; and
detecting, by the one or more detection assemblies, the emission light emitted by the sample.

46. The method of claim 45, further comprising:
processing, at a computing device, the detected emission light to generate one or more sets of spectral and/or spatial responses, each set representing a spatial distribution of a corresponding component in the one or more components over the area of the sample.

47. The method of claim 45 or 46, wherein the sample is blood or urine.
